(19) United States
(12) Patent Application Publication
(10) Pub. No.: US 2021/0038122 A1
(43) Pub. Date: Feb. 11, 2021

(54) METHOD FOR DETECTING BODY MOVEMENTS OF A SLEEPING PERSON

(71) Applicant: AIT AUSTRIAN INSTITUTE OF TECHNOLOGY GMBH, WIEN (AT)

(72) Inventors: BERNHARD KOHN, DEUTSCH-BRODERSDORF (AT); MARKUS GALL, WIEN (AT); CHRISTOPH WIESMEYR, WIEN (AT); HEINRICH GARN, WIEN (AT); MARKUS WASER, WIEN (AT)

(21) Appl. No.: 16/963,909

(22) PCT Filed: Jan. 21, 2019
(86) PCT No.: PCT/AT2019/060019

§ 371 Date: Jul. 22, 2020

(30) Foreign Application Priority Data
Jan. 22, 2018 (AT) A50049/2018

(51) Int. Cl. A61B 5/11; A61B 5/00

(52) U.S. Cl. CPC A61B 5/1128; A61B 5/4812; A61B 5/706; A61B 5/6889; A61B 5/7267; A61B 5/6891; A61B 5/7203

(57) ABSTRACT

A method for detecting body movements of a sleeping person includes continuously creating three-dimensional images using an imaging unit and providing distance measurement values pixel by pixel. A two or three-dimensional height profile has points on a surface, or on, or next to the person. The profile is stored and available for each time. A range indicating a body part or area depending on a reference point or range is selected as a first region of interest. Periods of changes of the profile of the first region exceeding a first threshold value, and intervals therebetween are determined. Noise values of the profile are determined pixel by pixel for the intervals. Further changes of the profile exceeding a second threshold value in the intervals are determined, considering the noise value pixel by pixel. The periods and further periods of changes of the profile are registered as body movements.

The invention relates to a method for detecting body movements of a sleeping person in accordance with the preamble of patent claim 1.

The prior art discloses various methods for monitoring sleeping persons, in particular also for detecting movements during the sleep of persons, where it is possible in principle to identify pathological conditions of the sleeping person and to process them accordingly. One significant problem with such monitoring systems is that movements during the sleep occur only rarely and the complete capturing of the behavior of a person during the sleep typically leads to large quantities of data, of which a multiplicity can be discarded owing to the lack of movement of the person during sleep. Moreover, the individual movements during sleep are of varying quality and can have different degrees of intensity. It is therefore an object of the invention to reliably and easily detect the individual movements of a person occurring during sleep, in particular including at times when the relevant person is covered by a blanket, as is typically the case during sleep. Since the average person is typically covered by a blanket during sleep, capturing without a blanket, which is more accurate in measurement terms, can lead to distortions of the test result, with the result that, overall, it is desirable to capture a sleeping person covered by a blanket so as to be able to monitor the person in a sleep state that is as natural as possible.

The invention achieves this object by a method of the type mentioned in the introductory part having the characterizing feature of patent claim 1.

Here, provision is made in particular that
a) a two-dimensional or three-dimensional height profile of the person is created,
   wherein, in the height profile, a number of at least two points located on the surface of the person or on the surface of an object situated on or next to the person is defined in space, and
   wherein the respective height profile for each of the recording time points is stored and held available in a data structure,
   wherein an area denoting a specified body part or body area of the person in dependence on a reference point or reference area is selected as a first region of interest,
b) time ranges of changes in the height profile of the first region of interest the extent of which exceeds a specified first threshold value and also time gaps between said time ranges are determined,
c) a noise level value of the height profile is determined in a pixel-wise manner for each of the time gaps,
d) further time ranges of changes in the height profile the extent of which exceeds a second threshold value are determined in the time gaps while taking into account the respective noise level value in a pixel-wise manner, and
e) in each case the time ranges identified in steps b) and d) and further time ranges of changes in the height profile are registered as body movements of the person.

It is additionally a significant advantage of the procedure illustrated here that even relatively slight body movements during sleep can be distinguished from the noise due to the measurement arrangement or the measurement devices associated with the three-dimensional capturing and movements can be easily and efficiently recognized.

Two particularly advantageous further developments of the invention with which different representation forms of the height profile can be obtained make provision that, in step a), the height profile has a number of at least two distance measurement values for defining in each case one point in space, wherein the individual distance measurement values in each case denote the distance of the intersection point of a beam, which has been fixed in advance relative to the detector unit ascertaining the distance measurement values and in particular emanates from the detector unit, with the surface of the person or the surface of an object situated on or next to the person from a reference point or a reference plane,
b) wherein for each of the recording time points one data structure is created in each case that contains the respective height profile, wherein all the data structures thus created in each case have the same size and in each case have storage positions for the individual distance measurement values of the height profile.

Alternatively, provision may be made that
the height profile is characterized by a two-dimensional matrix data structure comprising a number of rows and columns,
a number of positions that are arranged in rows and columns in the manner of a grid and at which in each case the distance measurement values of the height profile are determined are specified,
the matrix data structure has a grid of the same size and structure, and
the matrix data structure is created by storing and holding available the distance measurement values recorded at the respective positions at the storage positions in the matrix data structure that correspond to the positions in the grid,
wherein a number of storage positions of the data structure, in which distance measurement values that denote the distance of a specified body part or body area of the person in dependence on the respective reference point or reference area are stored, is defined as a first region of interest in the height profile.

A particularly advantageous detection of changes in the height profile makes provision that in each case one movement map is created in each case for individual time points in step b) by forming, in an element-wise or pixel-wise manner, a local temporal change extent value as an extent value for the change of the individual distance measurement values of the points of the height profile in the first region of interest.

Alternatively, provision may be made that, for the same purpose, in each case a movement map for the first region of interest is created in each case for individual time points in step b) by way of, possibly weighted, accumulation or subtraction, in particular in a pixel-wise or element-wise manner, to the distance measurement values of the points of the height profile that were ascertained within one time interval around the respective time point in the respective pixel.

A particularly advantageous creation of a function that denotes the intensity of the movements of the person over time makes provision that a specified function is applied in step b) to specified elements of the movement map of the first region of interest and that an accumulation is performed, time point by time point, over the obtained values of the movement map and in this way a temporal movement function g(t) is obtained.

Provision may in particular be made here that, for creating said temporal function, a summation is performed over the first region of interest as the accumulation over the obtained values of the movement map and the temporal movement function g(t) is thus obtained.

Particularly advantageously, the presence of the temporal function characterizing the person's movements to pixel-wise threshold value exceedances or pixel-wise threshold value comparisons can be used for the detection of a threshold value exceedance.

Provision can be made here that a function is applied to the individual values of the movement map before the accumulation, wherein said function provides a threshold value comparison to a specified threshold value, and that the function returns a zero value if the value falls below the threshold value and, if it exceeds the threshold value,
returns a specified value,
returns the argument or the respective value of the movement map, or
returns the extent by which the respective value of the movement map has exceeded the threshold value.

Moreover, it is likewise possible to provide a pattern comparison for identifying changes in the height profile. In this case, provision is specifically made that, in step b), a pattern comparison or a threshold value comparison is performed in the temporal movement function g(t) to identify changes in the height profile of the first region of interest, wherein time ranges during which the temporal movement function g(t) corresponds to a specified pattern or exceeds a specified threshold value are recognized as being time ranges with changes.

A particularly advantageous type of detection, compensation and noise of individual sensors when creating the height profile and advantageous dealing with noisy measurement values of the height profile makes provision that, in step c), a noise map is created for each of the time gaps by ascertaining, in a pixel-wise manner, the noise of the individual distance measurement values in a second region of interest,

in particular wherein the standard deviation of the distance measurement value is ascertained within the respective time period in each case for individual time intervals and an average value of all the standard deviations thus ascertained within the time period is ascertained and used as the value of the noise map for the respective pixel.

Individual advantageous procedures for noise compensation specifically make provision that, in a first step, the ascertained distance values are weighted with a weighting value and in this way normalized distance measurement values are created, which is indirectly proportional to the noise value ascertained for the respective pixel in the noise map, and,

in a second step,
in each case a further movement map is created in each case for individual time points by the pixel-wise formation of an extent for the temporal change of the normalized distance measurement values in the second region of interest, and/or
in each case a further movement map for the second region of interest is created in each case for individual time points by pixel-wise, possibly weighted, accumulation to the normalized distance measurement values of the points of the height profile ascertained within a time interval around the respective time point in the respective pixel.

A particularly advantageous creation of a function that denotes the intensity of the movements of the person over time makes provision that a specified function is applied to specified points of the further movement map of the second region of interest and an accumulation over the obtained values of the further movement map is performed in a time-point-wise manner and in this way a further temporal function g′(t) is obtained.

Provision may be made in particular here, for creating said temporal function, that a summation over the second region of interest is performed as the accumulation over the obtained values of the further movement map and the further temporal function g′(t) is obtained in this way.

Particularly advantageously, the presence of the temporal function characterizing the person's movements to pixel-wise threshold value exceedances or pixel-wise threshold value comparisons can be used for the detection of a threshold value exceedance.

Provision may be made here that a function is applied to the individual values of the further movement map before the accumulation, wherein said function provides a threshold value comparison to a specified further threshold value, and that the function returns a zero value if the value falls below the threshold value and, if it exceeds the threshold value,
returns a specified value,
returns the argument or the respective value of the further movement map, or
returns the extent by which the argument or the respective value of the further movement map has exceeded the threshold value.

Moreover, it is likewise possible to provide a pattern comparison for identifying changes in the height profile. In this case, provision is specifically made that, in step d), a pattern comparison or a threshold value comparison is performed in the further temporal function g′(t) to identify changes in the height profile of the second region of interest, wherein time ranges during which the further temporal function g′(t) corresponds to a specified pattern or exceeds a specified second threshold value are recognized as being further time ranges with changes.

The areas of interest can be defined, within the framework of steps b) and c), advantageously by virtue of the fact
that the further region of interest is greater than the first region of interest, and/or
that the further region of interest contains the first region of interest, and/or
that the further region of interest corresponds to the first region of interest.

In order to pre-set specific body areas that are known in advance and are relevant for the examination in question, provision may be made that the first region of interest and/or the further region of interest are defined in advance in the height profile, in particular such that the first region of interest and/or the further region of interest contain areas of the height profile that correspond to specified areas of the body of the person.

In order to make it possible to define body regions of interest in an automated manner, provision may be made that a body model is specified and regions of interest in the height profile are defined in an automated manner by
a) searching for areas that correspond to specified body parts or to a specified body region in a recording of the person using the body model and an object classification algorithm and defining the identified areas or areas derived therefrom as regions of interest, or
b) searching for an area that corresponds to the head of the person for a number of time points in the respective recording of the person in a pixel-wise manner using the body model and an object classification algorithm and thus determining the location of the body of the person in the respective recording, wherein the regions of interest are defined in relation to the ascertained location of the body, in particular at a specified distance from the head.

The temporal adaptation of the region of interest can be controlled by only using or also using assignments from recordings of the person that were created temporally before the recording time point of the respectively considered recording for the pixel-wise assignment of areas of the respectively considered recording to a body part or a body region.

Efficient assignment of the individual movements to individual body parts provides
that every recording is divided into a specified number of grid elements,
that, for each grid element, a method as claimed in one of claims 1 to 14 is carried out, wherein the respective grid element is defined as the region of interest, and
that the grid element in question is assigned to a body part in the case of detecting changes of the height profile in the respective grid element using the body model and a movement of said body part is ascertained.

A few preferred exemplary embodiments of the invention that should be understood to be nonlimiting will be illustrated on the basis of the following drawings.

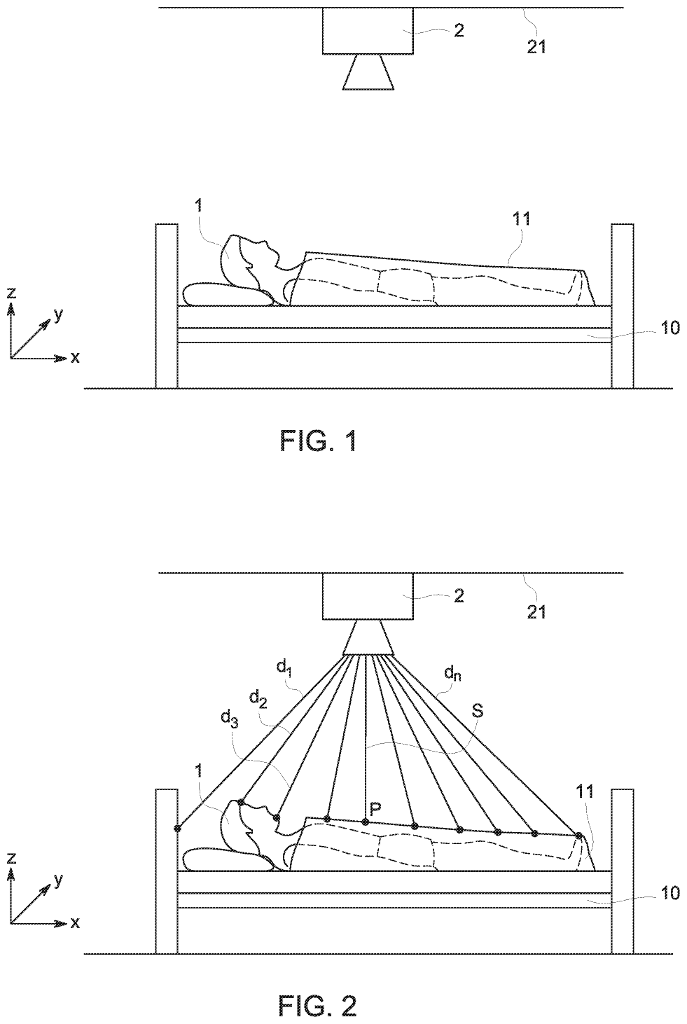

FIG. 1 shows an arrangement for capturing and detecting body movements of a sleeping person.

FIGS. 2, 3 and 4 show different definitions of height profiles.

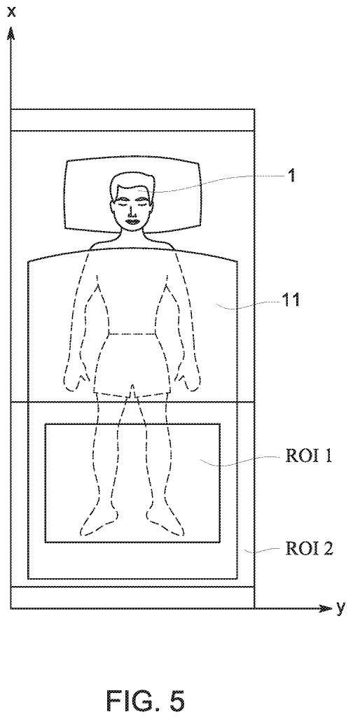

FIG. 5 schematically shows a height profile in the form of an image.

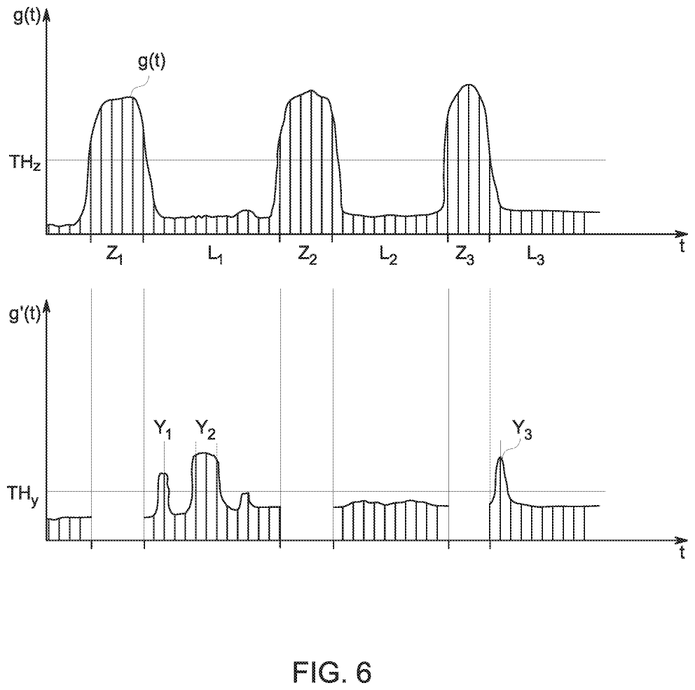

FIG. 6 schematically shows the movement function, the further movement function and the analysis thereof for ascertaining body movements.

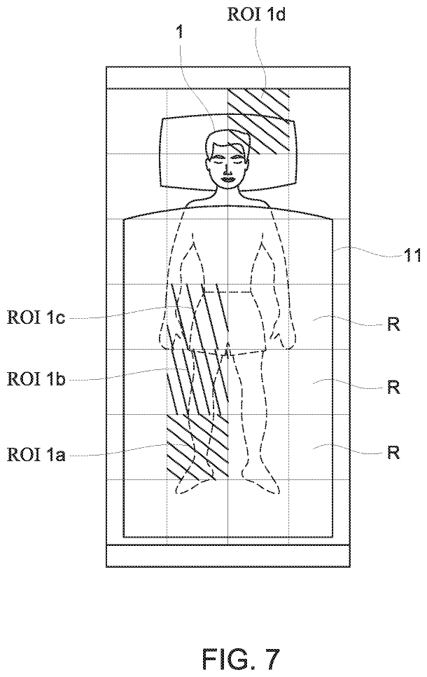

FIG. 7 schematically shows some possibilities for defining regions of interest.

FIG. 1 shows an arrangement for capturing and detecting body movements of a sleeping person 1 from the side. This arrangement can be typically used in sleep laboratories or similar medical monitoring devices. In this case, the person 1 is intended to be examined for the presence of specific sleep disorders and is monitored for this purpose as they are lying in a bed 10, sleeping. The person may also be at least partially covered by a blanket to increase comfort during sleep.

An image recording unit 2, configured for creating three-dimensional recordings of the person 1, is arranged above the person 1. Said three-dimensional recordings are created as part of a recording step a) typically in the form of a height profile H (FIG. 4), which has in each case one sampling value for a multiplicity of different beams. The height profile that is present can be recorded for example by recording, as is illustrated in FIG. 2, in each case separately distance measurement values d₁, . . . , dₙ, which denote the distance of the intersection point P of the surface of the person 1 with the beam S from the image recording unit 2 along the specified beam S for a number of beams S that are coming from the image recording unit 2, are arranged in the manner of a grid, and emanate from the image recording unit 2.

In the height profile H, a number of at least two points, preferably of a multiplicity of points P located on beams S that are arranged in the manner of a grid, is generally defined in space, said points P being located on the surface of the person 1 or on the surface of an object situated on or next to the person 1, such as the blanket 11 or the bed 10.

The height profile H can thus be defined by virtue of the fact that, as is illustrated in FIG. 2, the distance measurement values d₁, d₂, . . . , dₙ of the height profile H are defined as the distances between the points P, located on the beams S, on the surface of the person 1 and the image recording unit 2. The distances can here be measured in different ways, for example using a 3D camera.

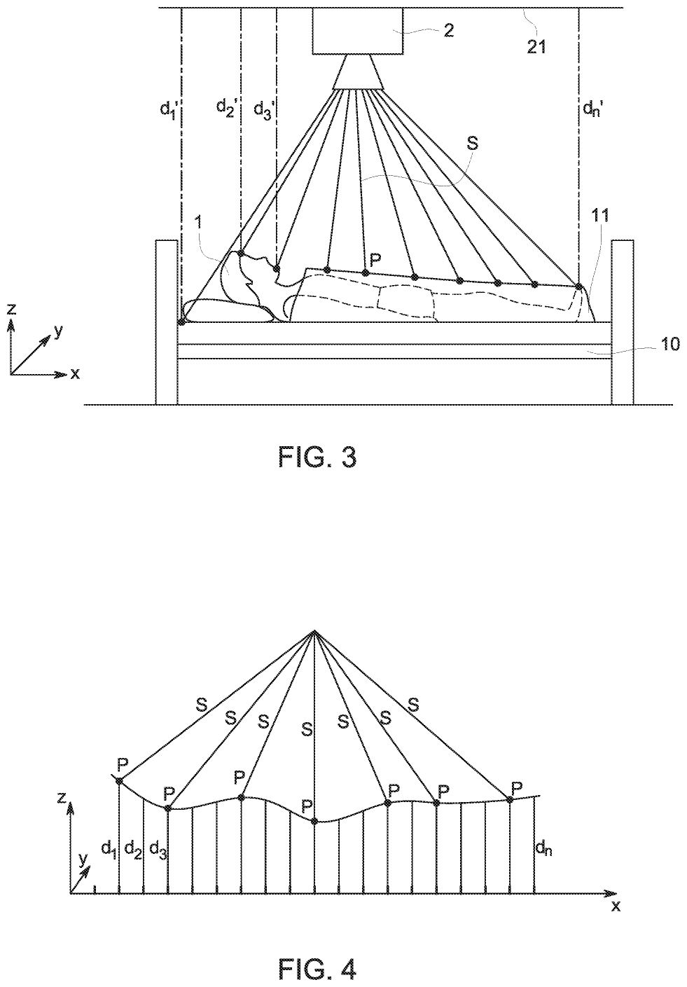

Alternatively, the individual distance measurement values d₁′, d₂′, dₙ′ can, as is illustrated in FIG. 3, be defined when the height profile is created as the distances between the ascertained points P on the surface of the person 1 and another object, in particular the ceiling 21 of the examination room.

It is likewise possible, as is illustrated in FIG. 4, to find for the individual points an interpolating curve that is used as the height profile H. This interpolating curve can likewise be evaluated at a multiplicity of three-dimensional points, with the result that, for a number of x- and y-coordinate values arranged in the form of a grid, in each case a z-coordinate value that likewise lies on the curve is made available.

For individual recording time points, in each case a separate height profile H is ascertained here and stored in a data structure. This height profile H, which is illustrated schematically in FIG. 4, has in each case one distance measurement value d₁, . . . , dₙ for a number of elements or pixels that are arranged in the manner of a grid or an image, wherein the distance measurement values can be defined as described above.

As is illustrated in FIG. 5, a region of interest ROI1, in which typically parts of the person 1 whose movement is to be monitored are situated, is selected within the height profile. Since the objective in the present case is to monitor the movements of the legs belonging to the person 1, the region of interest is located in the lower portion of the height profile. If, on the other hand, other body regions or other regions withing the height profile H are to be monitored, a corresponding different selection of the height profile can be made.

However, it is possible in principle for the selection of the region of interest ROI1 to be defined manually and to include the body regions whose movements are intended to be monitored in concrete terms.

If in each case one height profile H is ascertained at each of the recording time points, a data structure containing the respective height profile H is available in each case for each individual one of the recording time points t₁, . . . , tₚ. All the data structures thus created have among themselves in each case the same size and have storage positions for the individual ascertained distance measurement values d₁, . . . , dₙ of the height profile.

The height profile can be created particularly easily if the individual beams S emanating from the detector unit are arranged in the form of a grid and each of the distance measurement values is entered in a matrix data structure that constitutes the grid of the individual beams S or has a structure that corresponds to the structure of the grid of the individual beams S. For example, if the grid contains 300×300 beams, the matrix data structure has 300×300 entries which, if the content of the matrix data structure is considered an image, can also be referred to as pixels.

At each of the recording time points t₁, . . . , tₚ, in each case a separate matrix data structure is created here by virtue of the fact that the distance measurement values d₁, . . . , dₙ recorded at the respective positions can be stored and held available at the storage positions in the matrix data structure that correspond to the positions in the grid.

The storage positions of the data structure in which distance measurement values d₁, . . . , dₙ are stored that are situated in the region of interest of the height profile, are analogously also referred to as region of interest ROI1 of the data structure.

It is possible in this way to create individual height profiles of the sleeping person or the surface of the sleeping person 1 or the blanket 11 covering said person 1 or the bed 10 situated next to the person 1 for a multiplicity of time points. Consequently there is a data structure from which the significant movements of the sleeping person 1 can be taken.

In the first processing step b) following the recording step a) illustrated here, time ranges Z₁, . . . , Z₃ of changes in the height profile H within the first region of interest, in which the extent for the temporal change of the height profile H exceeds a specified first threshold value, are captured. Furthermore, the time gaps L₁, L₂, . . . between said time ranges Z₁, . . . Z₃ are ascertained.

Defining and determining the extent for the temporal change in the height profile H can be effected here in different ways. One particularly easy variant makes provision in this context that in each case a movement map MM1 is created for individual time points, in particular for all time points t₁, . . . , tₚ, by ascertaining in an element-wise or pixel-wise manner for each distance measurement value d₁, . . . , dₙ or each visual ray S or each entry k(x, y, t) of the matrix data structure a local change extent value mm(x, y, t) for the temporal change in the respective distance measurement value d₁, . . . , dₙ or the entry k(x, y, t) entered in the respective data structure.

Once a first movement map MM1 has been created in this way for a number of time points t₁, . . . , tₚ, it is possible to ascertain for each individual time point t₁, . . . , tₚ in each case a movement value that is ascertained by accumulation over the obtained local change extent values mm(x, y, t) of the movement map MM1 within the region of interest ROI1.

When determining an accumulated movement value for a specific time point that is entered in the temporal movement function g(t), in principle all the available local change extent values mm(x, y, t) or the local change extent values mm(x, y, t) that are formed in different ways can be used. In particular, it is also possible that the individual local change extent values mm(x, y, t) of the movement map MM1 within the region of interest ROI1 are ascertained in a pixel-wise manner, wherein individual entries k(x, y, t), assigned to the same pixel or element of the data structure, or change extent values that lie within a specified time interval around the respective time point t₁, . . . , tₚ, are added in a weighted manner. For example, it is thus possible that for determining a local change extent value mm(x, y, t) in a specified pixel or entry at the grid position x, y of the region of interest ROI1 is determined, in which the relevant distance measurement values last recorded or entries k(x, y, t) are added in a weighted manner, wherein the individual weights can be defined in different ways.

In the simplest case, the temporal change can be ascertained for example by subtraction of the two distance measurement values or entries k(x, y, t); k(x, y, t−1), which were ascertained at the same position at immediately successive time points. If appropriate, it is also possible, as long as the type of movement is irrelevant, for the absolute value of the difference of the two entries k(x, y, t); k(x, y, t−1) or distance measurement values to be used as the local change extent value mm(x, y, t) for the relevant entry or the relevant relevant pixel at the position x, y at the time point t. For the determination of the change extent value mm(x, y, t), it is likewise also possible that a number of entries k(x, y, t), which were in each case recorded within a time interval before the respective time point t in the same pixel at the position x, y, is used and an average value km₁(x, y, t) or the, possibly weighted, sum km₁(x, y, t) is ascertained from said entries k(x, y, t). A number of entries k(x, y, t) recorded in each case within a time interval after the respective time point t in the same pixel at the position x, y, is likewise used and an average value km₂(x, y, t) or the, possibly weighted, sum km₂(x, y, t) is ascertained from said entries k(x, y, t). Subsequently, the change value is formed by formation of the difference of the two average values thus created or sums km₁(x, y, t); km₂(x, y, t) and is used as the change extent value mm(x, y, t).

A particularly simple possibility for determining an accumulated overall extent for the temporal change in the height profile H at a time point t₁, . . . , tₚ for the region of interest ROI1 can be effected for example by summation or addition of all local change extent values mm(x, y, t) that are contained at a time point or in a movement map MM1(t). In addition, other procedures for accumulation can also be chosen, in particular a function h can be applied to the individual values of the movement map MM1(t) before the summation.

This function h(x) can have different configurations. In the present case, functions that contain a threshold value comparison and compare the respective movement value to a specified threshold value TH₁ are recommended in particular. If the value falls below said threshold value TH₁, the relevant function h(x) can return a zero value, which makes no contribution to the accumulation, in particular the value 0. However, if the value exceeds the threshold value TH₁, the function h(x) can return different values; in particular, the function can return a specified constant value, such as 1, which makes a contribution to the accumulation and does not correspond to the zero value. After applying an addition, a function value for the temporal movement function g(t) is obtained, which corresponds to the number of those pixels or entries in which in each case a threshold value exceedance has been ascertained.

In addition, there are also other possibilities for defining the function h(x); for example, it is also possible for the case of the exceedance of the threshold value TH₁ by the argument of the function h(x) to return the argument, i.e. in the concrete case the respective value of the movement map MM1, with the function h thus having the following form, for example:
h(x)={if x<TH₁, then 0, otherwise x}

Moreover, the function h(x) can also be defined such that, if the threshold value TH₁ is exceeded, it is not the argument x itself but the extent of the exceedance of the threshold value TH₁ by the argument x or by the respective value of the movement map MM1 that is returned:
h(x)={if x<TH₁, then 0, otherwise x-TH₁}

One example of the profile of the accumulated change value or for a movement function g(t) is illustrated in FIG. 6. FIG. 6 furthermore shows the ascertainment of time ranges during which body movements occur. In principle, the temporal movement function g(t) can be compared to a specified threshold value TH_{Z} to detect time ranges Z₁, Z₂, Z₃ with significant changes or changes exceeding a threshold value. If the movement function g(t) exceeds the relevant threshold value TH_{Z}, the time range during which the movement function g(t) exceeds the relevant threshold value TH_{Z} is identified as a time range Z₁, Z₂, Z₃ of significant changes in the height profile H or in movements of the person 1 and is held available as such.

Alternatively, it is also possible that it is not the concrete extent of the temporal movement function g(t) but the occurrence of specific patterns in the temporal movement function g(t) that is considered to be conclusive for the presence of relevant movements of the person 1. In this case, different pattern comparisons can be performed and time ranges Z₁, Z₂, Z₃, in which the temporal movement function g(t) corresponds to the respective specified pattern, can be identified as time ranges Z₁, Z₂, Z₃ with significant changes or movements of the person 1.

In the first processing step b) illustrated above, individual time ranges in which in each case movements or changes in the height profile for the relevant person 1 occur were identified. It will now be illustrated below how it is possible to identify further movements of the person 1 that are likewise relevant in the time periods between the time ranges Z₁, Z₂, Z₃. It should be noted here in particular that a further decrease of the threshold value TH_{z} could possibly lead to a situation in which, on account of an increased noise of the recording unit 2, the decrease of the threshold value TH_{z} leads to a multiplicity of measurement results that do not represent movements of the person 1 that exceed a threshold value but are merely caused by the sensor noise of the image recording unit 2 that is unavoidably present.

To avoid said noise-related artifacts, a noise value r(x, y; L₁); r(x, y; L₂); r(x, y; L₃) of the height profile H is ascertained in a pixel-wise manner in a following step c) in the temporal gaps L₁, L₂, L₃ between the time ranges Z₁, Z₂, Z₃. This pixel-wise ascertainment of the noise value r(x, y, L) is not performed separately at each individual time point but in each case for an overall temporal gap L₁, L₂, L₃.

Overall, a number of noise values r(x, y; L₁) in the form of a noise map RM(L₁) is available after said calibration for a second region of interest ROI2, which can correspond in particular to the first region of interest ROI′ but can also be larger than the first region of interest or can contain the first region of interest. The noise values r(x, y; L₁) of the noise map noise map RM(L₁) can correspond for example to the standard deviation of the respective distance measurement value determined in each case separately for each pixel or for each entry starting from the individual positions or of the entries k(x, y, t) within the respective temporal gap L₁. For each temporal gap L₁, L₂, L₃, there is available in each case separately a noise map RM(L₁), RM(L₂), RM(L₂). It is likewise possible to ascertain in each case the standard deviation of the distance measurement values (d₁, . . . , dₙ) or of the entries of the data structure for each of the pixels within the temporal gap L₁, L₂, L₃ for individual overlapping or successive time intervals in the respective gap L₁, L₂, L₃. The average value of all standard deviations ascertained in this way within the gap L₁, L₂, L₃ is ascertained and used as the value r(x, y, L) of the noise map RM for the respective pixel.

After a noise map has been ascertained in the processing step c), in a further processing step d) the ascertained distance measurement values k(x, y, t) in the individual temporal gaps L₁, L₂, L₃ are weighted with a weight value that is indirectly proportional to the noise value r(x, y; L₁); r(x, y; L₂); r(x, y; L₃) ascertained for the respective pixel or the respective position x, y in the noise map RM(L₁), RM(L₂), RM(L₂) and in this way in each case a normalized distance measurement value e(x, y, t) is created in each case for each pixel or each entry of the data structure. In the simplest case, the respective normalized distance measurement value e(x, y, t) is ascertained by division of the respective distance measurement value e(x, y, t) by the noise value r(x, y; L₁) ascertained for the respective gap and the respective position.

Next, in each case a further movement map MM2(t) is created in each case for individual time points t within the temporal gaps L₁, L₂, L₃ by pixel-wise formation of a further local change extent value mm₂(x, y, t) by ascertaining the temporal change in the respective normalized distance measurement value e(x, y, t). The determination of the further movement map is created for the individual pixels or entries of the second region of interest ROI2.

Subsequently, a further temporal movement function g′(t) is created that corresponds to the temporal movement function g(t) but is created not on the basis of movement map MM1 but on the basis of the further movement map MM2. In this case, however, the same principles are used that were also used when creating the temporal movement function g(t).

Again, the individual further local change extent values mm₂(x, y, t) of the further movement map MM2 within the region of interest ROI2 can here be accumulated and an accumulation value obtained in this way can be allocated to a further temporal movement function g′(t). The function h(x) used above to create the temporal movement function can also be used for weighting the individual further local change extent values mm₂(x, y, t), but wherein a different threshold value TH₂ can also be used rather than the threshold value TH₁.

It is subsequently possible to identify further time ranges Y₁, Y₂, Y₃ of changes in the height profile H that are due to body movements of the person 1 by way of an analysis of the further movement function g′(t) by way of a threshold value comparison with a threshold value TH_{Y} or by way of a pattern comparison.

In principle, the movements ascertained in the time ranges Z₁, Z₂, Z₃ and in the further time ranges Y₁, Y₂, Y₃ can be identified as body movements of the relevant person 1.

The concrete choice of the regions of interest ROI1, ROI2 can, as has already been mentioned, take place in principle in different ways; in particular, the relevant region ROI1, ROI2 can be chosen by selecting a region of interest ROI1, ROI2 within the bed at which the body parts of interest are typically situated during the normal sleep position.

The second region of interest ROI2 can preferably also be larger than the first region of interest ROI1 or contain the first region of interest ROI1. This has the advantage that, in such a case, the first region can be limited to sensors or distance values having noise that is typically low, which is the case in particular in the case of distance sensors at the center of the imaging area of the image recording unit 2. In the peripheral areas, in which the sensor noise can be greater, by contrast, there is the risk that threshold value exceedances caused by noise result in an overestimation of the movements or that the ascertained results can contain artifacts due to the sensor noise rather than to body movements. However, since normalized measurement values, i.e. measurement values free of noise, can be present in the processing steps c) and d), it is also possible to use sensor measurement values that overall have a greater noise content for further processing.

A further particularly preferred alternative definition of the regions of interest with which in particular head movements can be detected makes provision that an area that corresponds to the head of the person is searched for in a pixel-wise manner in each height profile H using the body model and an object classification algorithm and in this way the location of the body of the person 1 in the respective recording is determined. Starting from the location of the body that is thus ascertained, the areas in which the relevant body regions are situated can be defined as a region of interest ROI1 or regions ROI1a, ROI1b, . . .

It is furthermore alternatively possible to also search for areas that correspond to specified body parts or a specified body region in a pixel-wise manner using a body model and an object classification algorithm in each recording of the person or in individual recordings of the person. The areas in which the identified areas are situated can subsequently be defined as regions of interest ROI1a, ROI1b, . . . , ROI1d.

The further region of interest ROI2 can be defined as described above also by equating the further region of interest ROI2 to the respective region of interest or by defining it as containing the latter.

In a preferred embodiment of the invention illustrated in FIG. 7, the height profile H is divided into a multiplicity of different tile-shaped grid elements R, wherein each grid element R is preferably rectangular in the height profile H and in each case corresponds to a potential region of interest ROI1a, ROI1b, ROI1c, ROI1d. For each individual grid element of interest R, there is the possibility of ascertaining separately in each case, by applying the method according to the invention illustrated above, whether body movements are located in each case within the grid element that are relevant overall.

If movements have been detected in the height profile H at the relevant grid element R, a body model and an object classification algorithm are applied to the relevant grid element and it is ascertained which body part is depicted in the relevant grid element. Subsequently, this recognized body part is ascertained as moving.

Even if the individual recording steps are performed successively in principle, it is possible to process height profiles that have also already been ascertained parallel to the recording of individual height profiles H in the recording step a). In particular, it is also possible to process the relevant height profiles in real time or with only a little time delay; in particular, it is possible to perform all processing steps for the respectively temporal gap L₁, L₂, L₃ found last if the time range Z₁, Z₂, Z₃ of changes terminating the respective gap L₁, L₂, L₃ was recognized in each case. In this case, it is already possible to ascertain the noise value for the respective gap L₁, L₂, L₃ and to determine the further time ranges Y₁, Y₂, Y₃. This has in particular the significant advantage that the respective physician monitoring the person can quickly be informed of the occurrence of movements during sleep or that here only slight delay times occur between the occurrence of the movement and the physician being informed.

1-20. (canceled)

21. In a method for detecting body movements of a sleeping person which includes continuously creating three-dimensional recordings of the person at successive recording time points with an image recording unit aimed at the person and providing ascertained distance measurement values, the improvement comprising:
a) creating a two-dimensional or three-dimensional height profile of the person;
defining at least two points located on a surface of the person or on a surface of an object situated on or next to the person in space in the height profile;
storing and holding available in a data structure a respective height profile for each of the recording time points; and
selecting an area denoting a specified body part or body area of the person in dependence on a reference point or reference area as a first region of interest;
b) determining time ranges of changes in the height profile of the first region of interest having an extent exceeding a specified first threshold value as well as time gaps between the time ranges;
c) determining a noise level value of the height profile in a pixel-wise manner for each of the time gaps;
d) determining further time ranges of changes in the height profile having an extent exceeding a second threshold value in time gaps while taking into account a respective noise level value in a pixel-wise manner; and
e) registering each of the time ranges identified in steps b) and d) and further time ranges of changes in the height profile as body movements of the person.

22. The method according to claim 21, which further
comprises: in step a), providing the height profile with at least two distance measurement values each defining one respective point in space, the individual distance measurement values each denoting a distance of an intersection point of a beam having been fixed in advance relative to a detector unit ascertaining the distance measurement values and emanating from the detector unit, with the surface of the person or the surface of an object situated on or next to the person from a reference point or a reference plane; and
in step b), creating a respective data structure containing the respective height profile for each of the recording time points, all of the data structures thus created having an identical size and each having respective storage positions for the individual distance measurement values of the height profile.

23. The method according to claim 21, which further comprises: characterizing the height profile by a two-dimensional matrix data structure
including a plurality of rows and columns;
specifying a plurality of positions disposed in rows and columns as a grid and at which the distance measurement values of the height profile are determined;
providing the matrix data structure with a grid having an identical size and structure;
creating the matrix data structure by storing and holding available the distance measurement values recorded at the respective positions at the storage positions in the matrix data structure corresponding to the positions in the grid; and
defining a first region of interest in the height profile as a plurality of storage positions of the data structure, in which distance measurement values denoting a distance of a specified body part or body area of the person in dependence on the respective reference point or reference area are stored.

24. The method according to claim 23, which further comprises creating one movement map for each respective individual time point in step b) by forming, in an element-wise or pixel-wise manner, a local temporal change extent value as an extent value for a change of the individual distance measurement values of the points of the height profile in the first region of interest.

25. The method according to claim 23, which further comprises creating a movement map for the first region of interest for each respective individual time point in step b) by way of, possibly weighted, accumulation or subtraction, in a pixel-wise or element-wise manner, to the distance measurement values of the points of the height profile having been ascertained within one time interval around the respective time point in a respective pixel.

26. The method according to claim 24, which further comprises, in step b), applying a specified function to specified elements of the movement map of the first region of interest and performing an accumulation, time point by time point, over obtained values of the movement map to obtain a temporal movement function g.

27. The method according to claim 26, which further comprises performing a summation over the first region of interest as the accumulation over the obtained values of the movement map to obtain the temporal movement function g.

28. The method according to claim 24, which further comprises applying a function to the individual values of the movement map before the accumulation, the function providing a threshold value comparison to a specified threshold value, the function returning a zero value when the value falls below the threshold value and, when the value exceeds the threshold value:
returning a specified value,
returning an argument or the respective value of the movement map, or returning an extent by which the respective value of the movement map has exceeded the threshold value.

29. The method according to claim 24, which further comprises, in step b), performing a pattern comparison or a threshold value comparison in the temporal movement function g to identify changes in the height profile of the first region of interest, and recognizing time ranges during which the temporal movement function g corresponds to a specified pattern or exceeds a specified threshold value as being time ranges with changes.

30. The method according to claim 23, which further comprises:
in step c), creating a noise map for each of the time gaps by ascertaining, in a pixel-wise manner, a noise of the individual distance measurement values in a second region of interest; and
ascertaining a standard deviation of the distance measurement value within the respective time period for each respective individual time interval and ascertaining an average value of all of the ascertained standard deviations within the time period and using the average value as a value of the noise map for the respective pixel.

31. The method according to claim 30, which further comprises:
in a first step, weighting the ascertained distance values with a weighting value to create normalized distance measurement values being indirectly proportional to the noise value ascertained for the respective pixel in the noise map; and
in a second step at least one of:
creating a further movement map for each respective individual time point by a pixel-wise formation of an extent for the temporal change of the normalized distance measurement values in the second region of interest, or
creating a further movement map for the second region of interest for each respective individual time point by pixel-wise, possibly weighted, accumulation to the normalized distance measurement value, of the points of the height profile ascertained within a time interval around the respective time point in the respective pixel.

32. The method according to claim 31, which further comprises applying a specified function to specified points of the further movement map of the second region of interest and performing an accumulation over the obtained values of the further movement map in a time-point-wise manner to obtain a further temporal function g′.

33. The method according to claim 32, which further comprises performing a summation over the second region of interest as the accumulation over the obtained values of the further movement map to obtain the further temporal function g′.

34. The method according to claim 31, which further comprises applying a function to the individual values of the further movement map before the accumulation, the function providing a threshold value comparison to a specified further threshold value, the function returning a zero value when the value falls below the threshold value and, when the value exceeds the threshold value:
returning a specified value,
returning an argument or a respective value of the further movement map, or
returning an extent by which the argument or the respective value of the further movement map has exceeded the threshold value.

35. The method according to claim 32, which further comprises, in step d), performing a pattern comparison or a threshold value comparison in the further temporal function g′ to identify changes in the height profile of the second region of interest, and recognizing time ranges during which the further temporal function g′ corresponds to a specified pattern or exceeds a specified second threshold value as being further time ranges with changes.

36. The method according to claim 30, wherein the further region of interest at least one of:
is larger than the first region of interest, or contains
the first region of interest, or corresponds to the first region of interest.

37. The method according to claim 30, which further
comprises: defining at least one of the first region of interest or the further region of interest in advance in the height profile; and
providing areas of the height profile corresponding to specified areas of the body of the person in at least one of the first region of interest or the further region of interest.

38. The method according to claim 21, which further comprises specifying a body model and defining regions of interest in the height profile in an automated manner by:
a) searching for areas corresponding to specified body parts or a specified body region in a recording of the person using the body model and an object classification algorithm and defining the identified areas or areas derived therefrom as regions of interest, or
b) searching for an area corresponding to the head of the person for a plurality of time points in the respective recording of the person in a pixel-wise manner using the body model and an object classification algorithm to determine a location of the body of the person in the respective recording, and defining regions of interest in relation to an ascertained location of the body at a specified distance from the head.

39. The method according to claim 38, which further comprises only using or also using assignments from recordings of the person having been created temporally before the recording time point of the respectively considered recording for a pixel-wise assignment of areas of the respectively considered recording to a body part or a body region.

40. The method according to claim 21, which further comprises: dividing every recording into a specified plurality of grid elements;
carrying out the method for each grid element and defining a respective grid element as a region of interest; and
assigning a grid element to a body part upon detecting changes of the height profile in the respective grid element using the body model and ascertaining a movement of the body part.

41. The method according to claim 21, which further comprises providing the ascertained distance measurement values in a pixel-wise manner.
